(19) United States
(12) Patent Application Publication
(10) Pub. No.: US 2009/0044397 A1
(43) Pub. Date: Feb. 19, 2009

(54) OPHTHALMIC PUMP ASSEMBLY

(76) Inventors: BEN Z. COHEN, New York, NY (US); NIGEL B. KELLY, Rye Brook, NY (US)

Correspondence Address: HOFFMANN & BARON, LLP, 6900 JERICHO TURNPIKE, SYOSSET, NY, 11791, US

(21) Appl. No.: 12/169,344

(22) Filed: Jul. 8, 2008

Related U.S. Application Data
(60) Provisional application No. 60/948,509, filed on Jul. 9, 2007.

(51) Int. Cl. B23P 11/00; A61F 9/00

(52) U.S. Cl. 29/428; 604/294

(57) ABSTRACT

In a first aspect of the subject invention, a method is provided for preparing a pump assembly which includes providing a glass or plastic eyedrop vial having a predetermined ophthalmic fluid therein; providing a pump mechanism having a pump inlet; and, securing the pump mechanism to the vial with the pump inlet extending into the vial to form a pump assembly. With the subject invention, a pump assembly can be advantageously formed using standard glass or plastic eyedrop vials. In a second aspect of the subject invention, a method is provided of preparing a pump assembly which includes providing an eyedrop dispenser having a dropper portion and a vial; removing the dropper portion from the vial; providing a pump mechanism having a pump inlet; and, securing the pump mechanism to the vial with the pump inlet extending into the vial to form a pump assembly. With the subject invention, a standard eyedrop ophthalmic dispenser may be retrofitted with a pump mechanism and converted into a pump-type ophthalmic dispenser.

### CROSS-REFERENCE TO RELATED APPLICATION

This application claims priority to U.S. Provisional Patent Application No. 60/948,509, filed Jul. 9, 2007, the entire contents of which are hereby incorporated by reference.

### BACKGROUND OF THE INVENTION

This invention relates to ophthalmic dispensers, and, more particularly, to ophthalmic pump assemblies.

Ophthalmic dispensers are well known in the prior art. The most common form of ophthalmic dispensers are eyedrop ophthalmic dispensers which deliver ophthalmic liquid in falling drop form and include a vial and a dropper portion. The most common form of an eyedrop ophthalmic dispenser is a dropper bottle which includes a vial having a deflectable wall which is squeezed to urge out one or more drops through a nozzle mounted to the neck of the vial (also known as a droptainer). The nozzle meters drops of the target liquid in acting as the dropper portion. In addition, eyedrop ophthalmic dispensers may include eyedroppers which are provided with a vial and a screw-on cap to which is mounted a dropper having a rubber squeeze bulb for administering one or more drops of ophthalmic liquid. With this arrangement, a glass vial may be utilized since deflection of the vial is not necessary for dose administration. Certain ophthalmic fluids are not stable in plastic and require glass for stable storage.

In addition to eyedrop ophthalmic dispensers, pumps also have been developed in the prior art for dispensing ophthalmic fluid. An example of such a pump is disclosed in U.S. Pat. No. RE38,077.

### SUMMARY OF THE INVENTION

In a first aspect of the subject invention, a method is provided for preparing a pump assembly which includes providing a glass or plastic eyedrop vial having a predetermined ophthalmic fluid therein; providing a pump mechanism having a pump inlet; and, securing the pump mechanism to the vial with the pump inlet extending into the vial to form a pump, assembly. With the subject invention, a pump assembly can be advantageously formed using standard glass or plastic eyedrop vials.

In a second aspect of the subject invention, a method is provided of preparing a pump assembly which includes providing an eyedrop dispenser having a dropper portion and a vial; removing the dropper portion from the vial; providing a pump mechanism having a pump inlet; and, securing the pump mechanism to the vial with the pump inlet extending into the vial to form a pump assembly. With the subject invention, a standard eyedrop ophthalmic dispenser may be retrofitted with a pump mechanism and converted into a pump-type ophthalmic dispenser.

These and other features of the invention will be better understood through a study of the following detailed description and accompanying drawings.

### BRIEF DESCRIPTION OF THE DRAWINGS

FIGS. 1-2A depict a dropper bottle ophthalmic dispenser which includes a deflectable dropper-bottle vial and dropper portion;

FIGS. 3-4 depict an eyedropper-type ophthalmic dispenser which includes a vial with screw-on cap to which is mounted a dropper portion;

FIG. 5 depicts a pump assembly formed in accordance with the subject invention; and,

FIG. 6 depicts an enlarged view of a connection useable between a pump mechanism and a vial in accordance with the subject invention.

### DETAILED DESCRIPTION OF THE INVENTION

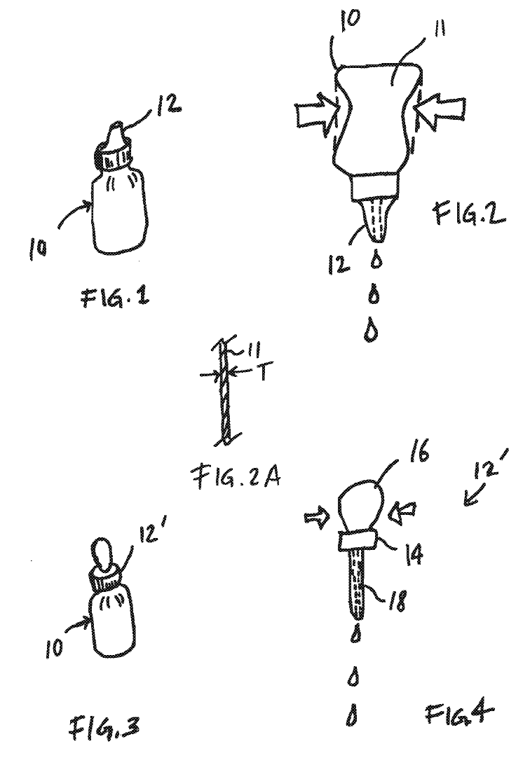

With reference to FIGS. 1-4, eyedrop ophthalmic dispensers are shown, which deliver ophthalmic liquid in falling drop form. FIGS. 1 and 2 depict a common dropper bottle dispenser (also referred to as a “droptainer”) which includes a vial 10 and a dropper portion 12. In a dropper bottle configuration as shown in FIGS. 1-2, the vial 10 is formed from a deflectable material, such as plastic, so as to be squeezable to urge out drops of ophthalmic fluid from the dispenser portion 12, as shown in FIG. 2. The vial 10 may be formed of various flexible materials to permit sufficient inward deflection to urge out doses from the vial 10. The vial 10 may be formed of a single wall construction, particularly a single side wall 11 (FIG. 2A), with a side wall thickness T of 0.032 inches or less. A thinner wall thickness T imparts greater wall flexibility. The vial 10 may be formed of various thermoplastics including, but not limited to, polyethylene terephthalate (PET), polypropylene (including syndiotactic polypropylene (SPP)), high density polyethylene (HDPE), low density polyethylene (LDPE), and combinations thereof.

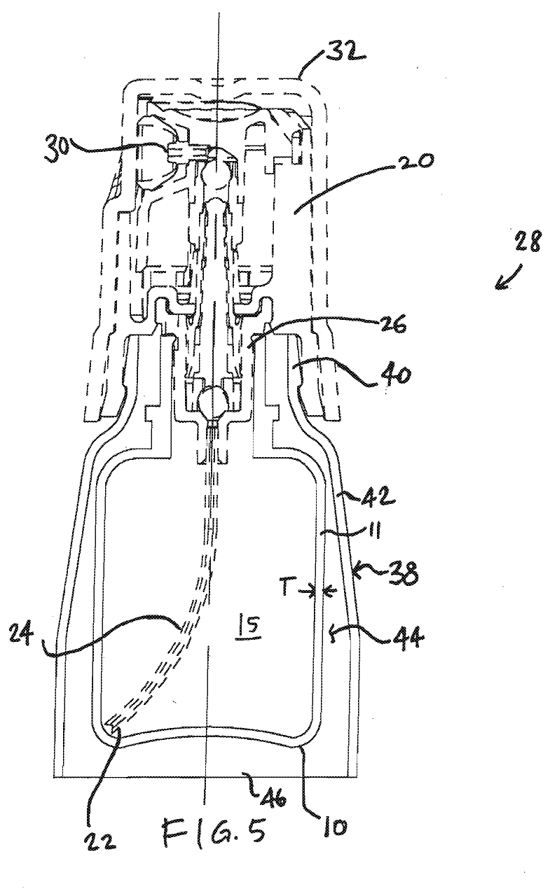

FIGS. 3 and 4 show the vial 10, with a second type of dropper portion 12′. The dropper portion 12′ includes a screw-on cap 14 which can be threaded and unthreaded on the vial 10. A dispensing bulb 16, which is deformable, is provided for urging drops of liquid out of shaft 18. The dropper portion 12′ must be removed from the vial 10 to administer doses. Since deflection of the vial 10 is not required for dose administration with the dropper portion 12′, the vial 10 may be formed from a rigid material, including glass. Optionally, the vial 10 may be formed from a thermoplastic material, including one or more of the materials listed above. The vial 10 may be formed with thicker wall thickness than noted above since side wall deflection is not a concern where the dropper portion 12′ is utilized. It is noted that certain ophthalmic liquids may be better stored in glass, than plastic, over prolonged periods. The vial 10 may be formed transparent, tinted, partially opaque, or fully opaque.

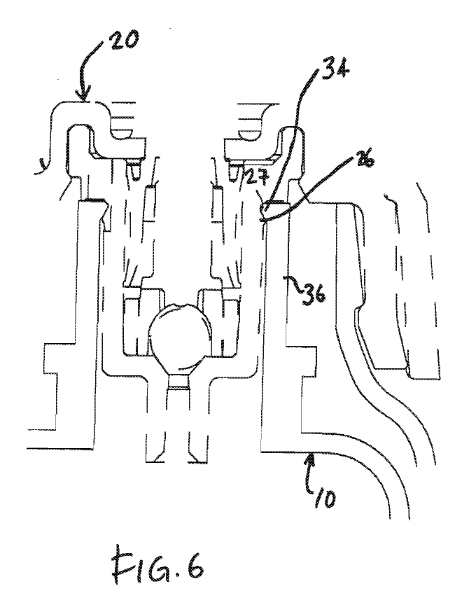

In any form, the vial 10 includes a reservoir 15 (FIG. 5) which may be formed to accommodate different volumes, including being formed to accommodate a volume of: 5-30 ml, including a volume of 5 ml, 10 ml, 15 ml, 20 ml, 25 ml, or 30 ml. The reservoir 15 may accommodate various liquids, particularly ophthalmic fluids, which will typically include one or more pharmaceutically-active agent(s).

With reference to FIGS. 5 and 6, a pump mechanism 20 is provided which includes a pump inlet 22 that may be defined in any known manner, including at the end of a dip tube 24. Any known pump mechanism may be utilized for the pump mechanism 20, including the designs depicted in any of: U.S. Pat. No. 5,152,435; U.S. Pat. No. RE38,077; U.S. Pat. No. 6,513,682, U.S. Pat. No. 6,776,309; U.S. Pat. No. 6,991,137; U.S. Pat. No. 7,014,068; U.S. Pat. No. 7,073,733; U.S. Pat. No. 7,131,559; and, U.S. Pat. No. 7,207,486. All of the aforementioned references are incorporated by reference herein. The pump mechanism 20 is configured to deliver one or more doses of ophthalmic liquid to a user. The dose(s) are ejected from the pump mechanism 20 with momentum generated by the pump mechanism 20 and are not delivered by sole falling action (as with eyedrop ophthalmic dispensers).

The pump mechanism 20 is provided with one or more connecting members 26 formed to cooperate with a portion of the vial 10 in permitting securement of the pump mechanism 20 to the vial 10. Preferably, the interface between the vial 10 and the pump mechanism 20 defines an at least liquid-tight seal. This seal may be fluid tight.

The pump mechanism 20 and the vial 10 collectively define a pump assembly 28, as shown in FIG. 5. The pump mechanism 20 is configured to have the pump inlet 22 extend into the vial 10 with the pump mechanism 20 being secured to the vial 10. In this manner, the pump mechanism 20 may urge ophthalmic liquid located within the vial 10 through the inlet 22 and out of a discharge aperture 30 to be administered to a patient. The components of the pump assembly 28, particularly those in contact with the liquid being administered, need to be compatible with the liquid. An overcap 32 may be provided for the pump assembly 28, which is detachably mountable to the pump mechanism 20, to maintain cleanliness of the discharge aperture 30.

With reference to FIG. 6, the connecting member 26 may be an annular protrusion formed to cooperatively engage a bead 34 formed on neck 36 of the vial 10. A trough 27 may be formed to accommodate the bead 34 in enhancing the strength of the securement between the vial 10 and the pump mechanism 20. The interengagement of the connecting member 26 and the bead 34 restricts removal of the pump mechanism 20 from the vial 10. Various configurations may be utilized. In addition, detachable disengagement may be desired to permit multiple uses of the pump mechanism 20 with various vials. Accordingly, the connecting member 26 and the bead 34 may be formed to deflect and give way upon a threshold amount of separating force being applied to extract the pump mechanism 20 from the vial 10. Alternatively, the connecting; member 26 and the bead 34 may be formed to irreversibly lock or an additional locking feature may be provided. It is preferred that a liquid-tight seal be formed between the pump mechanism 20 and the vial 10 at the connection between the two components.

The pump mechanism 20 and the vial 10 may rely on a mechanical attachment (e.g., friction fit; interference fit), chemical attachment (e.g., fusion bonding), and/or adhesive connection to maintain the two components together. Re-usability of the pump mechanism 20 may be an issue with a releasable connection being provided. The pump assembly 28 may be formed disposable with no releasability between the pump mechanism 20 and the vial 10.

In use, the vial 10 may be filled with a predetermined ophthalmic fluid. With the vial 10 being formed of glass or of a deflectable material, such as that used with conventional dropper bottles (e.g., plastic), the pump mechanism 20 may be connected to the vial 10 to form the pump assembly 28. The pump mechanism 20 may be mounted to the vial 10 with little or no modification thereof.

In addition, the pump mechanism 20 may be utilized to retrofit an eyedrop ophthalmic dispenser, such as those shown in FIGS. 1-4. To achieve retrofit, the dropper portion 12, 12′ is removed from the vial 10 and the pump mechanism 20 is secured to the vial 10. A patient may prefer the use of the pump mechanism to conventional eyedrop configurations. In this manner, the ophthalmic liquid may be delivered in a different manner from the eyedrop configuration (i.e., ejected from a pump with momentum versus sole falling action). The retrofit may be used with the vial 10 being formed of glass or plastic.

With reference to FIG. 5, to further enhance the comfort of a user, a handling shroud 38 may be provided. The handling shroud 38 may be formed to at least partially encircle the vial 10 and with a diameter which can be comfortably gripped by a user. With a diameter larger than the vial 10, the handling shroud 38 allows for relatively small diameter vials 10 to be utilized in the pump assembly 28 without the associated awkward handling of such small diameter vials. In a preferred construction, the handling shroud 38 includes a mounting neck 40 adapted to be secured to the pump mechanism 20. The mounting neck 40 may be formed unitarily with the pump mechanism 20 or may be formed to be removably attachable thereto. Sidewall 42 of the handling shroud 38 extends down from the mounting neck 40 to define a gripping surface for a user and to define an internal space 44 for accommodating the vial 10. The sidewall 42 may be at least partially tapered.

The handling shroud 38 also includes distal end 46 which is preferably sufficiently open to permit passage therethrough of the vial 10. In this manner, the pump mechanism 20 and the handling shroud 38 may be pre-packaged with the vial 10 being secured thereto afterwards. Alternatively, the handling shroud 38 may be cup-shaped, with the distal end 46 being closed. The distal end 46 may be closed by a separate component, e.g., a disc snap-fitted onto the sidewall 42, or the handling shroud 38 may be unitarily formed with the distal end 46 being closed. With a closed configuration, the vial 10 must be mounted before or with the handling shroud 38 to the pump mechanism 20. This arrangement limits the ability to tamper with the vial 10.

1. A method of preparing a pump assembly, said method comprising the steps of:
providing a glass vial having a predetermined ophthalmic fluid therein;
providing a pump mechanism having a pump inlet; and,
securing said pump mechanism to said glass vial with said pump inlet extending into said glass vial to form a pump assembly.

2. A method as in claim 1, wherein said pump mechanism includes a handling shroud, said handling shroud at least partially encircling said glass vial with said glass vial secured to said pump mechanism.

3. A method of preparing a pump assembly, said method comprising the steps of:
providing a plastic dropper-bottle vial having a predetermined ophthalmic fluid therein, said dropper-bottle vial having a flexible sidewall with a thickness of 0.033 inches or less;
providing a pump mechanism having a pump inlet; and,
securing said pump mechanism to said plastic dropper-bottle vial with said pump inlet extending into said plastic dropper-bottle vial to form a pump assembly.

4. A method as in claim 3, wherein said pump mechanism includes a handling shroud, said handling shroud at least partially encircling said plastic dropper-bottle vial with said plastic dropper-bottle vial secured to said pump mechanism.

5. A method of preparing a pump assembly, said method comprising the steps of:
providing an eyedrop ophthalmic dispenser having a dropper portion and a vial;
removing said dropper portion from said vial;
providing a pump mechanism having a pump inlet; and,
securing said pump mechanism to said vial with said pump inlet extending into said vial to form a pump assembly.

6. A method as in claim 5, wherein said vial is formed of glass.

7. A method as in claim 5, wherein said vial is; formed of plastic.

8. A method as in claim 5, wherein said pump mechanism includes a handling shroud, said handling shroud at least partially encircling said vial with said vial secured to said pump mechanism.
